(19) United States
(12) Patent Application Publication
(10) Pub. No.: US 2011/0129566 A1
(43) Pub. Date: Jun. 2, 2011

(54) Functional Enhancement of Yeast to Minimize Production of Ethyl Carbamate Via Modified Transporter Expression

(75) Inventors: Hendrick J.J. Van Vuuren, Lions Bay (CA); John Ivan Husnik, Charlottetown (CA)

(73) Assignee: THE UNIVERSITY OF BRITISH COLUMBIA, VANCOUVER, B.C. (CA)

(21) Appl. No.: 12/937,892

(22) PCT Filed: Apr. 14, 2009
(86) PCT No.: PCT/CA09/00481

§ 371 Date: Feb. 7, 2011

Related U.S. Application Data
(60) Provisional application No. 61/071,138, filed on Apr. 14, 2008.

(51) Int. Cl. C12G 1/00; A23L 1/28; A23L 2/00; C12N 15/81; C12N 1/19

(52) U.S. Cl. 426/11; 426/60; 426/531; 426/590; 426/592; 435/471; 435/254.2

(57) ABSTRACT

The invention provides a Saccharomyces cerevisiae strain that is transformed to constitutively express DUR3, encoding a urea transporter protein, under the control of the phosphoglycerate kinase (PGK1) promoter and terminator sequences, resulting in reduced nitrogen catabolite repression, wherein said transformed yeast strain may be further transformed to constitutively express a urea degradation enzyme, such as urea carboxylase-allophanate hydrolase or urea amidolyase, also resulting in reduced nitrogen catabolite repression, a method for generating said strain, and a method and use of said strain to produce a fermented beverage or food product with a reduced ethyl carbamate concentration of less than 30 ppb.

### BACKGROUND

Ethyl Carbamate, also known as urethane, forms as a direct byproduct from the use of yeast to ferment foods and beverages. For example, the formation of ethyl carbamate, a probable carcinogen, occurs in fermenting grape must (wine) by reaction of urea with ethanol.

Yeast strains that degrade urea via constitutive expression of DUR1,2 may be used to produce a fermented beverage or food product with low ethyl carbamate concentrations (Coulon et al., 2006, Am. J. Enol. Vitic.: 113-124).

In Saccharomyces cerevisiae, the DUR3 gene encodes a urea transporter (DUR3p) that actively transports urea into the yeast cell under certain conditions. Transcription of the DUR3 gene is normally subject to Nitrogen Catabolite Repression (NCR, ElBerry et al., 1993, J. Bacteriol. 175: 4688-4698; Goffeau et al., 1996, Science 274 (5287), 546-547; Johnston et al., 1994, Science 265 (5181), 2077-2082). This is only one aspect of the regulatory network of anabolic and catabolic enzymes involved in nitrogen metabolism in a carbohydrate-utilizing yeast cell.

### SUMMARY

The invention relates, in part, to products and processes that provide for a reduction of ethyl carbamate concentration in a fermented beverage or food product, using a yeast strain that has been transformed to express a urea transporter, to actively transport urea into the yeast cell, such as DUR3, under fermenting conditions. The yeast may also be modified to express an intracellular urea degrading enzymatic activity under the fermenting conditions, such as DUR1,2.

The present invention provides, in part, a novel yeast strain which has been transformed to express DUR3 under fermenting conditions, for example constitutively, as well as methods for functional enhancement of yeast strains so that the yeast expresses DUR3 under fermenting conditions, for example constitutively, and the use of said yeast strains for the reduction of ethyl carbamate in a fermented beverage or food product.

In a another embodiment of the invention there is provided a novel yeast strain which has been transformed to constitutively express DUR1,2 and DUR3, and the use of said yeast strain for the reduction of ethyl carbamate in a fermented beverage or food product.

In a further embodiment of the invention there is provided a yeast strain transformed to continually express DUR3p and a yeast strain transformed to continually express both DUR3p and urea amidolyase containing both urea carboxylase and allophanate hydrolase activities.

In a further embodiment of the invention there is provided a yeast strain which has been transformed to continually uptake urea under fermenting conditions. Wherein said yeast may constitutively express DUR3.

In a further embodiment of the invention there is provided a yeast strain which has been transformed to continually uptake and also degrade urea under fermenting conditions. Wherein said yeast strain may constitutively express both DUR1,2 and DUR3.

In a further embodiment of the invention there is provided a method for modifying a yeast strain comprising transforming said yeast strain to reduce nitrogen catabolite repression of urea transporter expression under fermenting conditions. Wherein said urea transporter may be encoded by DUR3 and wherein said urea transporter may be DUR3p.

In a further embodiment of the invention there is provided a method for modifying a yeast strain to constitutively express DUR3. Wherein said method may include integration of the 1/2TRP1-PGKₚ-DUR3-PGKₜ-kanMX-1/2TRP1 cassette into the TRP1 locus. Wherein said method may include transforming said yeast strain with a novel nucleic acid comprising a coding sequence encoding DUR3p. Wherein said method may include transforming said yeast with a recombinant nucleic acid comprising a promoter not subject to nitrogen catabolite repression.

In a further embodiment of the invention there is provided a method of making a fermented beverage or food product by the use of a yeast strain functionally enhanced as described above, such as one that under fermenting conditions expresses DUR3, or both DUR1,2 and DUR3, for example by constitutive expression.

In a further embodiment of the invention there is provided the use of a transformed yeast strain that constitutively expresses DUR3, or both DUR1,2 and DUR3 to reduce the concentration of ethyl carbamate in a fermented beverage or food product. Wherein the fermented beverage or food product may be wine and the reduced concentration of ethyl carbamate may be below 30 ppb.

In a further embodiment of the invention there is provided a fermented beverage or food product having a reduced ethyl carbamate concentration produced using a transformed yeast strain that constitutively expresses DUR3, or both DUR1,2 and DUR3. Wherein the fermented beverage or food product may be wine and the reduced concentration of ethyl carbamate may be below 30 ppb.

### BRIEF DESCRIPTION OF THE DRAWINGS

FIG. 1. DUR3 genetic cassette

FIG. 2 sets out a S. cerevisiae DUR3p protein sequence (SEQ ID NO:7).

FIG. 3 sets out the S. cerevisiae DUR3 coding sequence (SEQ ID NO:8).

FIG. 4 sets out the sequence of a portion of the upstream region of the DUR1,2 gene, ending at the DUR1,2 start codon ATG (SEQ ID NO:9). Two putative NCR element GATAA(G) boxes are highlighted (one at position −54 to −58 and to other at position −320 to −324), as well as putative TATAA boxes.

FIG. 5 sets out the sequence of a portion of the upstream region of the DUR3 gene (SEQ ID NO:10).

FIG. 6 sets out a multiple protein sequence alignment, illustrating homologies between DUR3p (sequence NP_{—}011847.1 (SEQ ID NO:7)) and 7 other proteins (sequence NP_{—}595871.1 (SEQ ID NO:11), XP_{—}452980.1 (SEQ ID NO:12), NP_{—}982989.1 (SEQ ID NO:13), XP_{—}364218.1 (SEQ ID NO:14), XP_{—}329657.1 (SEQ ID NO:15), NP_{—}199351.1 (SEQ ID NO:16) and NP_{—}001065513.1 (SEQ ID NO:17)).

FIG. 7 illustrates a BLAST comparison of DUR3p (SEQ ID NO:7) with a (predicted) urea transporter of Schizosaccharomyces pombe (SEQ ID NO:11), and sets out a consensus sequence. In alternative embodiments, urea transporters of the invention may have various degrees of identity compared to the S. cerevisiae DUR3p sequence or to the S. pombe urea transporter, or to the consensus sequence set out in this Figure, such as 80% identity when optimally aligned.

FIG. 8 illustrates fermentation profiles (weight loss) of wine yeast strains 522, 522DUR1,2 [an alternative designation for 522^{EC-,}'], 522DUR3, and 522DUR1,2/DUR3 [an alternative designation for 522^{EC-DUR3}] in Chardonnay wine. Chardonnay wine was produced from unfiltered Calona Chardonnay must inoculated to a final OD600=0.1 and incubated to completion (˜300 hours) at 20° C. Fermentations were conducted in triplicate and data were averaged; error bars indicate the standard deviation.

FIG. 9 is a schematic illustration of a DUR3 self-cloning cassette of the invention.

FIG. 10 is a schematic representation of the integration of the self-cloning leu2-PGK1p-kanMX-PGK1p-DUR3-PGK1t-leu2 cassette into the LEU2 locus of S. cerevisiae industrial strains using a kanMX marker and subsequent loss of the marker by recombination of the PGK1 promoter direct repeats.

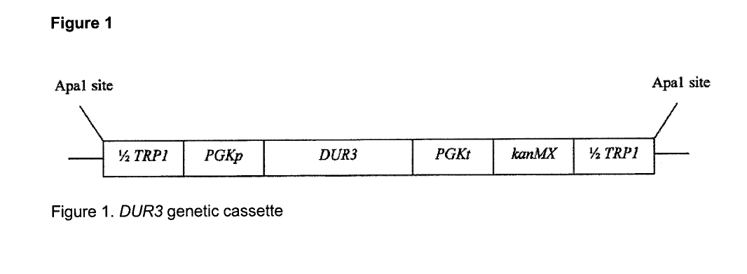

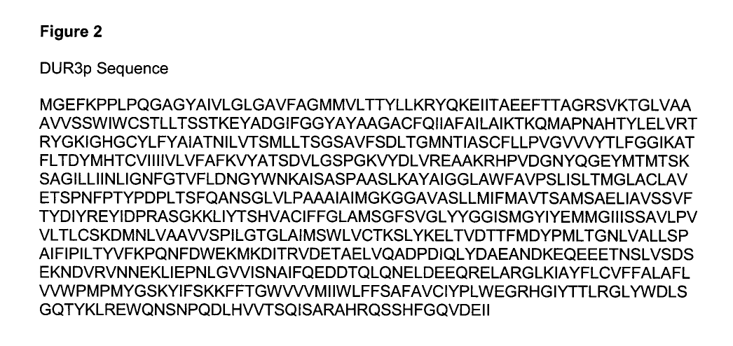

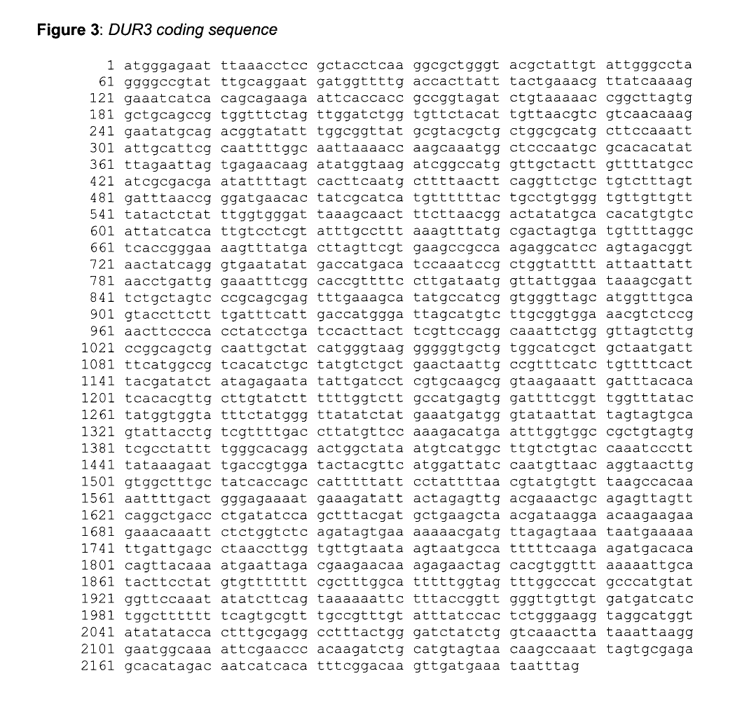

### DETAILED DESCRIPTION

Any terms not directly defined herein shall be understood to have the meanings commonly associated with them as understood within the art of the invention. Certain terms are discussed below, or elsewhere in the specification, to provide additional guidance to the practitioner in describing the compositions, devices, methods and the like of embodiments of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples in the specification, including examples of terms, is for illustrative purposes only and does not limit the scope and meaning of the embodiments of the invention herein.

As mentioned herein a ‘yeast strain’ may be a strain of Saccharomyces cerevisiae. In alternative embodiments, the invention may for example utilize S. bayanus yeast strains, or Schizosaccharomyces yeast strains.

In alternative aspects, the invention relates to yeast strains used in fermentation to produce a variety of products, such as a fermented beverage or food product. A ‘fermented beverage or food product’ may be, but is not limited to, wine, brandy, whiskey, distilled spirits, ethanol, sake, sherry, beer, dough, bread, vinegar, or soy sauce.

In various aspects, the present invention relates to the modification of genes and the use of recombinant genes. In this context, the term “gene” is used in accordance with its usual definition, to mean an operatively linked group of nucleic acid sequences. The modification of a gene in the context of the present invention may include the modification of any one of the various sequences that are operatively linked in the gene. By “operatively linked” it is meant that the particular sequences interact either directly or indirectly to carry out their intended function, such as mediation or modulation of gene expression. The interaction of operatively linked sequences may for example be mediated by proteins that in turn interact with the nucleic acid sequences.

In the context of the present invention, “promoter” means a nucleotide sequence capable of mediating or modulating transcription of a nucleotide sequence of interest in the desired spatial or temporal pattern and to the desired extent, when the transcriptional regulatory region is operably linked to the sequence of interest. A transcriptional regulatory region and a sequence of interest are “operably linked” when the sequences are functionally connected so as to permit transcription of the sequence of interest to be mediated or modulated by the transcriptional regulatory region. In some embodiments, to be operably linked, a transcriptional regulatory region may be located on the same strand as the sequence of interest. The transcriptional regulatory region may in some embodiments be located 5′ of the sequence of interest. In such embodiments, the transcriptional regulatory region may be directly 5′ of the sequence of interest or there may be intervening sequences between these regions. Transcriptional regulatory sequences may in some embodiments be located 3′ of the sequence of interest. The operable linkage of the transcriptional regulatory region and the sequence of interest may require appropriate molecules (such as transcriptional activator proteins) to be bound to the transcriptional regulatory region, the invention therefore encompasses embodiments in which such molecules are provided, either in vitro or in vivo.

Various genes and nucleic acid sequences of the invention may be recombinant sequences. The term “recombinant” means that something has been recombined, so that with reference to a nucleic acid construct the term refers to a molecule that is comprised of nucleic acid sequences that have at some point been joined together or produced by means of molecular biological techniques. The term “recombinant” when made with reference to a protein or a polypeptide refers to a protein or polypeptide molecule which is expressed using a recombinant nucleic acid construct created by means of molecular biological techniques. The term “recombinant” when made in reference to genetic composition refers to a gamete or progeny or cell or genome with new combinations of alleles that did not occur in the naturally-occurring parental genomes. Recombinant nucleic acid constructs may include a nucleotide sequence which is ligated to, or is manipulated to become ligated to, a nucleic acid sequence to which it is not ligated in nature, or to which it is ligated at a different location in nature. Referring to a nucleic acid construct as “recombinant” therefore indicates that the nucleic acid molecule has been manipulated by human intervention using genetic engineering.

Recombinant nucleic acid constructs may for example be introduced into a host cell by transformation. Such recombinant nucleic acid constructs may include sequences derived from the same host cell species or from different host cell species, which have been isolated and reintroduced into cells of the host species.

Recombinant nucleic acid sequences may become integrated into a host cell genome, either as a result of the original transformation of the host cells, or as the result of subsequent recombination and/or repair events. Alternatively, recombinant sequences may be maintained as extra-chromosomal elements. Such sequences may be reproduced, for example by using an organism such as a transformed yeast strain as a starting strain for strain improvement procedures implemented by mutation, mass mating or protoplast fusion. The resulting strains that preserve the recombinant sequence of the invention are themselves considered “recombinant” as that term is used herein.

In various aspects of the invention, nucleic acid molecules may be chemically synthesized using techniques such as are disclosed, for example, in Itakura et al. U.S. Pat. No. 4,598,049; Caruthers et al. U.S. Pat. No. 4,458,066; and Itakura U.S. Pat. Nos. 4,401,796 and 4,373,071. Such synthetic nucleic acids are by their nature “recombinant” as that term is used herein (being the product of successive steps of combining the constituent parts of the molecule).

Transformation is the process by which the genetic material carried by a cell is altered by incorporation of one or more exogenous nucleic acids into the cell. For example, yeast may be transformed using a variety of protocols (Gietz et al., 1995). Such transformation may occur by incorporation of the exogenous nucleic acid into the genetic material of the cell, or by virtue of an alteration in the endogenous genetic material of the cell that results from exposure of the cell to the exogenous nucleic acid. Transformants or transformed cells are cells, or descendants of cells, that have been functionally enhanced through the uptake of an exogenous nucleic acid. As these terms are used herein, they apply to descendants of transformed cells where the desired genetic alteration has been preserved through subsequent cellular generations, irrespective of other mutations or alterations that may also be present in the cells of the subsequent generations.

Transformed host cells for use in wine-making may for example include strains of S. cerevisiae or Schizosaccharomyces, such as Bourgovin (RC 212 Saccharomyces cerevisiae), ICV D-47 Saccharomyces cerevisiae, 71B-1122 Saccharomyces cerevisiae, K1V-1116 Saccharomyces cerevisiae, EC-1118 Saccharomyces bayanus, Vin13, Vin7, N96, and WE352. There are a variety of commercial sources for yeast strains, such as Lallemand Inc. (Canada), AB Mauri (Australia) and Lesaffre (France).

In various embodiments, aspects of the invention may make use of endogenous or heterologous enzymes having urea transport activity, such as the urea transport activity of DUR3. Similarly, in some embodiments, aspects of the invention may make use of endogenous or heterologous enzymes having urea degrading activity, such as the urea carboxylase and allophanate hydrolase activity of DUR1,2p. These enzymes may for example be homologous to DUR3p or DUR1,2p or to regions of DUR3p or DUR1,2p having the relevant activity.

The degree of homology between sequences (such as native DUR3p or DUR1,2p or native DUR3 or DUR1,2 nucleic acid sequences and the sequence of an alternative protein or nucleic acid for use in the invention) may be expressed as a percentage of identity when the sequences are optimally aligned, meaning the occurrence of exact matches between the sequences. Optimal alignment of sequences for comparisons of identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence alignment may also be carried out using the BLAST algorithm, described in Altschul et al., 1990, J. Mol. Biol. 215:403-10 (using the published default settings). Software for performing BLAST analysis may be available through the National Center for Biotechnology Information (through the internet at http://www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold. Initial neighbourhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST programs may use as defaults a word length (W) of 11, the BLOSUM62 scoring matrix (Henikoff and Henikoff, 1992, Proc. Natl. Acad. Sci. USA 89: 10915-10919) alignments (B) of 50, expectation (E) of 10 (which may be changed in alternative embodiments to 1 or 0.1 or 0.01 or 0.001 or 0.0001; although E values much higher than 0.1 may not identify functionally similar sequences, it is useful to examine hits with lower significance, E values between 0.1 and 10, for short regions of similarity), M=5, N=4, for nucleic acids a comparison of both strands. For protein comparisons, BLASTP may be used with defaults as follows: G=11 (cost to open a gap); E=1 (cost to extend a gap); E=10 (expectation value, at this setting, 10 hits with scores equal to or better than the defined alignment score, S, are expected to occur by chance in a database of the same size as the one being searched; the E value can be increased or decreased to alter the stringency of the search.); and W=3 (word size, default is 11 for BLASTN, 3 for other blast programs). The BLOSUM matrix assigns a probability score for each position in an alignment that is based on the frequency with which that substitution is known to occur among consensus blocks within related proteins. The BLOSUM62 (gap existence cost=11; per residue gap cost=1; lambda ratio=0.85) substitution matrix is used by default in BLAST 2.0. A variety of other matrices may be used as alternatives to BLOSUM62, including: PAM30 (9,1,0.87); PAM70 (10,1,0.87) BLOSUM80 (10,1,0.87); BLOSUM62 (11,1,0.82) and BLOSUM45 (14,2,0.87). One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

Nucleic acid and protein sequences of the invention may in some embodiments be substantially identical, such as substantially identical to DUR3p or DUR1,2p or DUR3 or DUR1,2 nucleic acid sequences. The substantial identity of such sequences may be reflected in percentage of identity when optimally aligned that may for example be greater than 50%, 80% to 100%, at least 80%, at least 90% or at least 95%, which in the case of gene targeting substrates may refer to the identity of a portion of the gene targeting substrate with a portion of the target sequence, wherein the degree of identity may facilitate homologous pairing and recombination and/or repair. An alternative indication that two nucleic acid sequences are substantially identical is that the two sequences hybridize to each other under moderately stringent, or preferably stringent, conditions. Hybridization to filter-bound sequences under moderately stringent conditions may, for example, be performed in 0.5 M NaHPO4, 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO4, 7% SDS, 1 mM EDTA at 65° C., and washing in 0.1×SSC/0.1% SDS at 68° C. (see Ausubel, et al. (eds), 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y.). Generally, stringent conditions are selected to be about 5° C. lower than the thermal melting point for the specific sequence at a defined ionic strength and pH. Washes for stingent hybridization may for example be of at least 15 minutes, 30 minutes, 45 minutes, 60 minutes, 75 minutes, 90 minutes, 105 minutes or 120 minutes.

It is well known in the art that some modifications and changes can be made in the structure of a polypeptide, such as DUR3 or DUR1,2, without substantially altering the biological function of that peptide, to obtain a biologically equivalent polypeptide. In one aspect of the invention, proteins having urea transport activity may include proteins that differ from the native DUR3 sequence by conservative amino acid substitutions. Similarly, proteins having urea carboxylase/allophanate hydrolase activity may include proteins that differ from the native DUR1,2 sequence by conservative amino acid substitutions. As used herein, the term “conserved amino acid substitutions” refers to the substitution of one amino acid for another at a given location in the protein, where the substitution can be made without substantial loss of the relevant function. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the protein by routine testing.

In some embodiments, conserved amino acid substitutions may be made where an amino acid residue is substituted for another having a similar hydrophilicity value (e.g., within a value of plus or minus 2.0), where the following may be an amino acid having a hydropathic index of about −1.6 such as Tyr (−1.3) or Pro (−1.6)s are assigned to amino acid residues (as detailed in U.S. Pat. No. 4,554,101, incorporated herein by reference): Arg (+3.0); Lys (+3.0); Asp (+3.0); Glu (+3.0); Ser (+0.3); Asn (+0.2); Gln (+0.2); Gly (O); Pro (−0.5); Thr (−0.4); Ala (−0.5); H is (−0.5); Cys (−1.0); Met (−1.3); Val (−1.5); Leu (−1.8); Ile (−1.8); Tyr (−2.3); Phe (−2.5); and Trp (−3.4).

In alternative embodiments, conserved amino acid substitutions may be made where an amino acid residue is substituted for another having a similar hydropathic index (e.g., within a value of plus or minus 2.0). In such embodiments, each amino acid residue may be assigned a hydropathic index on the basis of its hydrophobicity and charge characteristics, as follows: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); H is (−3.2); Glu (−3.5); Gln (−3.5); Asp (−3.5); Asn (−3.5); Lys (−3.9); and Arg (−4.5).

In alternative embodiments, conserved amino acid substitutions may be made where an amino acid residue is substituted for another in the same class, where the amino acids are divided into non-polar, acidic, basic and neutral classes, as follows: non-polar: Ala, Val, Leu, Ile, Phe, Trp, Pro, Met; acidic: Asp, Glu; basic: Lys, Arg, H is; neutral: Gly, Ser, Thr, Cys, Asn, Gln, Tyr.

In alternative embodiments, conservative amino acid changes include changes based on considerations of hydrophilicity or hydrophobicity, size or volume, or charge. Amino acids can be generally characterized as hydrophobic or hydrophilic, depending primarily on the properties of the amino acid side chain. A hydrophobic amino acid exhibits a hydrophobicity of greater than zero, and a hydrophilic amino acid exhibits a hydrophilicity of less than zero, based on the normalized consensus hydrophobicity scale of Eisenberg et al. (J. Mol. Bio. 179:125-142, 184). Genetically encoded hydrophobic amino acids include Gly, Ala, Phe, Val, Leu, Ile, Pro, Met and Trp, and genetically encoded hydrophilic amino acids include Thr, H is, Glu, Gln, Asp, Arg, Ser, and Lys. Non-genetically encoded hydrophobic amino acids include t-butylalanine, while non-genetically encoded hydrophilic amino acids include citrulline and homocysteine.

Hydrophobic or hydrophilic amino acids can be further subdivided based on the characteristics of their side chains. For example, an aromatic amino acid is a hydrophobic amino acid with a side chain containing at least one aromatic or heteroaromatic ring, which may contain one or more substituents such as —OH, —SH, —CN, —F, —Cl, —Br, —I, —NO2, —NO, —NH2, —NHR, —NRR, —C(O)R, —C(O)OH, —C(O)OR, —C(O)NH2, —C(O)NHR, —C(O)NRR, etc., where R is independently (C1-C6) alkyl, substituted (C1-C6) alkyl, (C1-C6) alkenyl, substituted (C1-C6) alkenyl, (C1-C6) alkynyl, substituted (C1-C6) alkynyl, (C5-C20) aryl, substituted (C5-C20) aryl, (C6-C26) alkaryl, substituted (C6-C26) alkaryl, 5-20 membered heteroaryl, substituted 5-20 membered heteroaryl, 6-26 membered alkheteroaryl or substituted 6-26 membered alkheteroaryl. Genetically encoded aromatic amino acids include Phe, Tyr, and Tryp.

An apolar amino acid is a hydrophobic amino acid with a side chain that is uncharged at physiological pH and which has bonds in which a pair of electrons shared in common by two atoms is generally held equally by each of the two atoms (i.e., the side chain is not polar). Genetically encoded apolar amino acids include Gly, Leu, Val, Ile, Ala, and Met. Apolar amino acids can be further subdivided to include aliphatic amino acids, which is a hydrophobic amino acid having an aliphatic hydrocarbon side chain. Genetically encoded aliphatic amino acids include Ala, Leu, Val, and Ile.

A polar amino acid is a hydrophilic amino acid with a side chain that is uncharged at physiological pH, but which has one bond in which the pair of electrons shared in common by two atoms is held more closely by one of the atoms. Genetically encoded polar amino acids include Ser, Thr, Asn, and Gln.

An acidic amino acid is a hydrophilic amino acid with a side chain pKa value of less than 7. Acidic amino acids typically have negatively charged side chains at physiological pH due to loss of a hydrogen ion. Genetically encoded acidic amino acids include Asp and Glu. A basic amino acid is a hydrophilic amino acid with a side chain pKa value of greater than 7. Basic amino acids typically have positively charged side chains at physiological pH due to association with hydronium ion. Genetically encoded basic amino acids include Arg, Lys, and His.

It will be appreciated by one skilled in the art that the above classifications are not absolute and that an amino acid may be classified in more than one category. In addition, amino acids can be classified based on known behaviour and or characteristic chemical, physical, or biological properties based on specified assays or as compared with previously identified amino acids.

In various aspects of the invention, the urea transport and degrading activity of a host may be adjusted so that it is at a desired level under fermentation conditions, such as under wine fermentation conditions. The term “fermentation conditions” or “fermenting conditions” means conditions under which an organism, such as S. cerevisiae, produces energy by fermentation, i.e. culture conditions under which fermentation takes place. Broadly defined, fermentation is the sum of anaerobic reactions that can provide energy for the growth of microorganisms in the absence of oxygen. Energy in fermentation is provided by substrate-level phosphorylation. In fermentation, an organic compound (the energy source) serves as a donor of electrons and another organic compound is the electron acceptor. Various organic substrates may be used for fermentation, such as carbohydrates, amino acids, purines and pyrimidines. In one aspect, the invention relates to organisms, such as yeast, capable of carbohydrate fermentation to produce ethyl alcohol.

In wine fermentation, the culture conditions of the must are derived from the fruit juice used as starting material. For example, the main constituents of grape juice are glucose (typically about 75 to 150 g/l), fructose (typically about 75 to 150 g/l), tartaric acid (typically about 2 to 10 g/l), malic acid (typically about 1 to 8 g/l) and free amino acids (typically about 0.2 to 2.5 g/l). However, virtually any fruit or sugary plant sap can be processed into an alcoholic beverage in a process in which the main reaction is the conversion of a carbohydrate to ethyl alcohol.

Wine yeast typically grows and ferments in a pH range of about 3.2 to 4.5 and requires a minimum water activity of about 0.85 (or a relative humidity of about 88%). The fermentation may be allowed to proceed spontaneously, or can be started by inoculation with a must that has been previously fermented, in which case the juice may be inoculated with populations of yeast of about 10⁶ to about 10⁷ cfu/ml juice. The must may be aerated to build up the yeast population. Once fermentation begins, the rapid production of carbon dioxide generally maintains anaerobic conditions. The temperature of fermentation is usually from 10° C. to 30° C., and the duration of the fermentation process may for example extend from a few days to a few weeks.

In one aspect, the present invention provides yeast strains that are capable of reducing the concentration of ethyl carbamate in fermented alcoholic beverages. For example, the invention may be used to provide wines having an ethyl carbamate concentration of less than 40 ppb (μg/L), 35 ppb, 30 ppb, 25 ppb, 20 ppb, 15 ppb, 10 ppb or 5 ppb (or any integer value between 50 ppb and 1 ppb). In alternative embodiments, the invention may be used to provide fortified wines or distilled spirits having an ethyl carbamate concentration of less than about 500 ppb, 400 ppb, 300 ppb, 200 ppb, 150 ppb, 100 ppb, 90 ppb, 80 ppb, 70 ppb, 60 ppb, 50 ppb, 40 ppb, 30 ppb, 20 ppb or 10 ppb (or any integer value between 500 ppb and 10 ppb).

In alternative embodiments, the invention may provide yeast strains that are capable of maintaining a reduced urea concentration in grape musts. For example, urea concentrations may be maintained below about 15 mg/l, 10 mg/l, 5 mg/l, 4 mg/l, 3 mg/l, 2 mg/l or 1 mg/l.

In one aspect, the invention provides methods for selecting natural mutants of a fermenting organism having a desired level of urea degrading activity under fermenting conditions. For example, yeast strains may be selected that lacking NCR of DUR3. For an example of mutation and selection protocols for yeast, see U.S. Pat. No. 6,140,108 issued to Mortimer et al. Oct. 31, 2000. In such methods, a yeast strain may be treated with a mutagen, such as ethylmethane sulfonate, nitrous acid, or hydroxylamine, which produce mutants with base-pair substitutions. Mutants with altered urea degrading activity may be screened for example by plating on an appropriate medium.

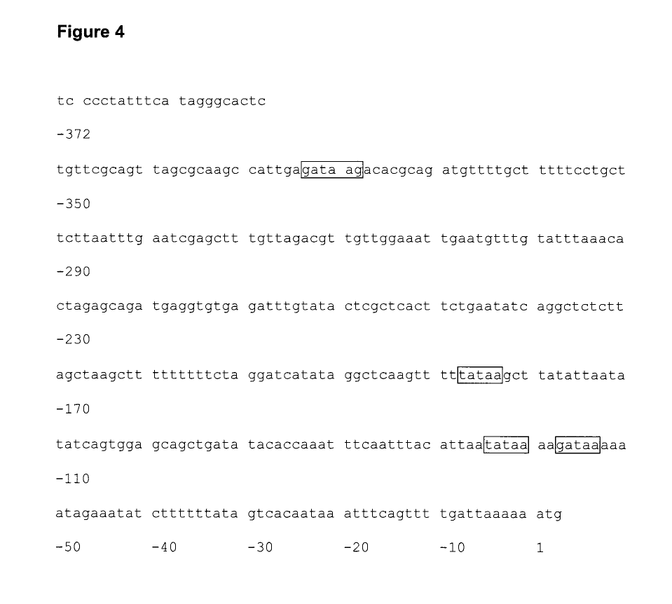

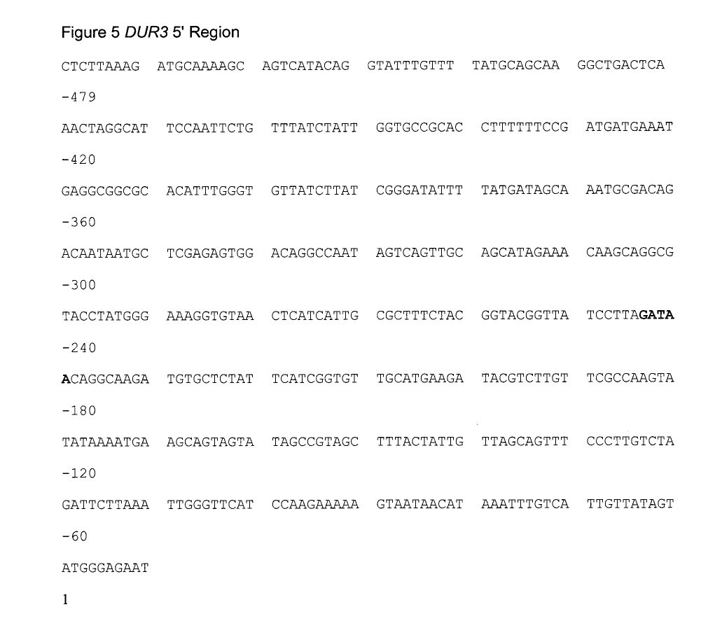

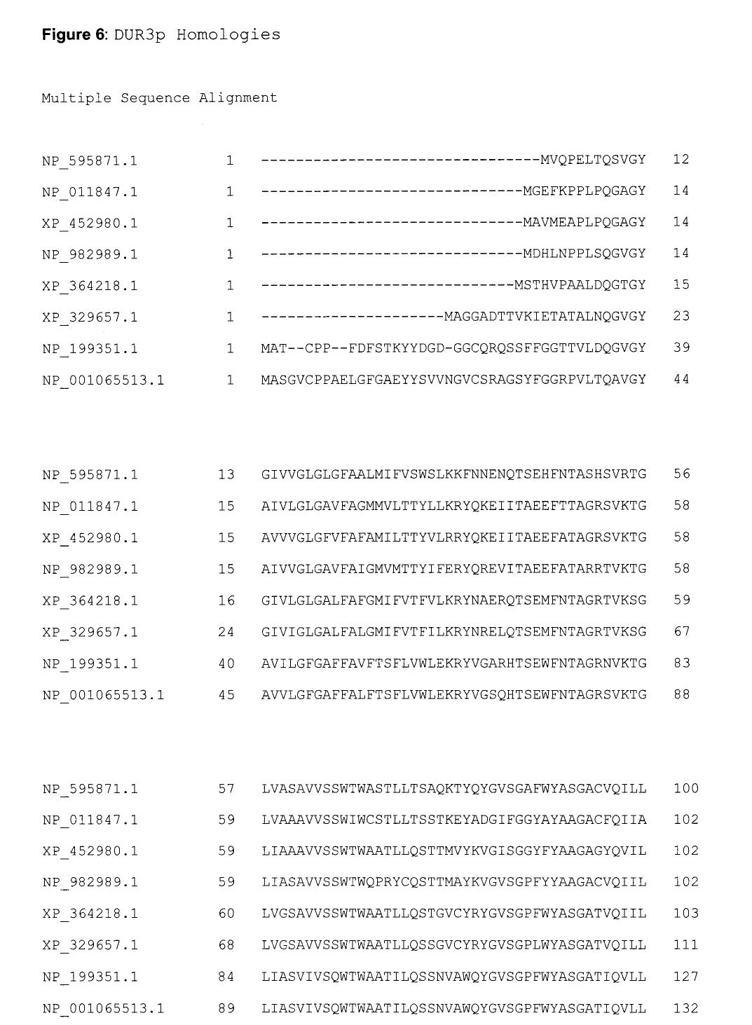

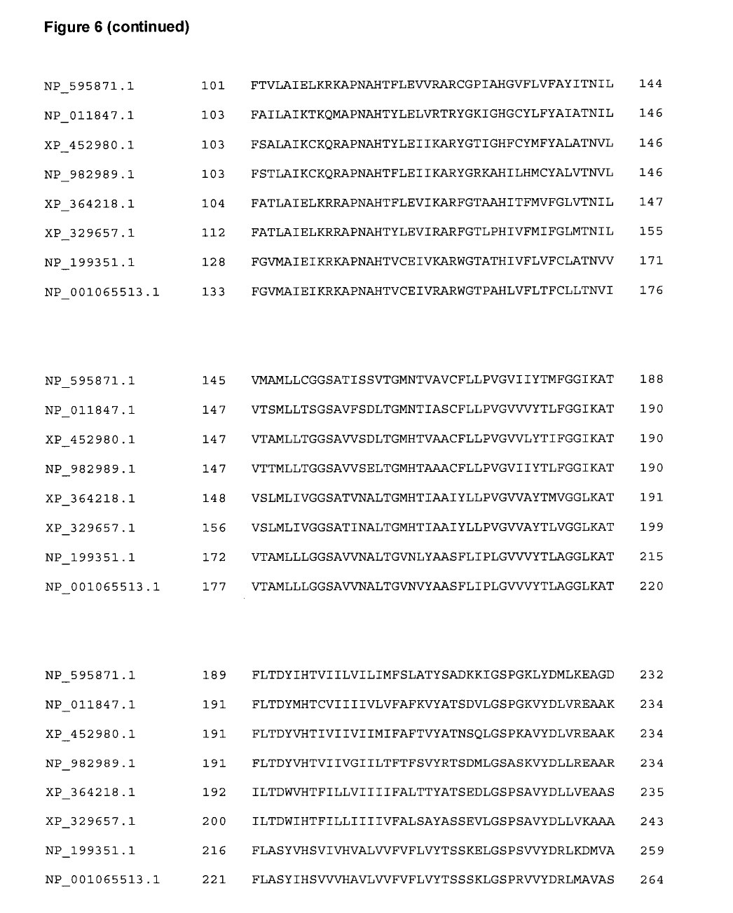

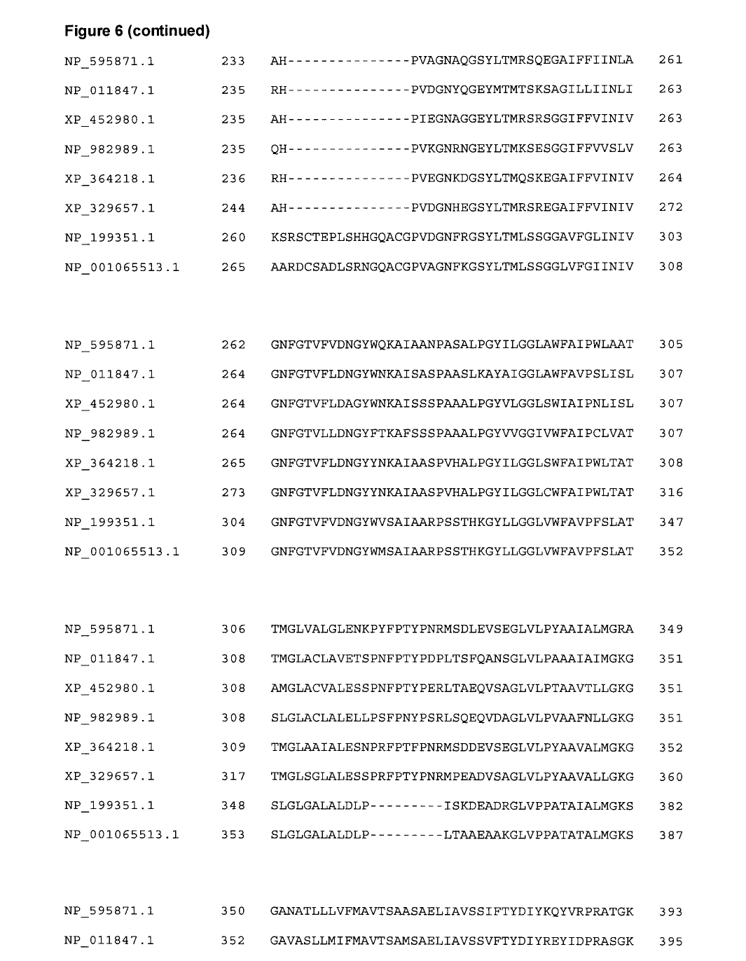

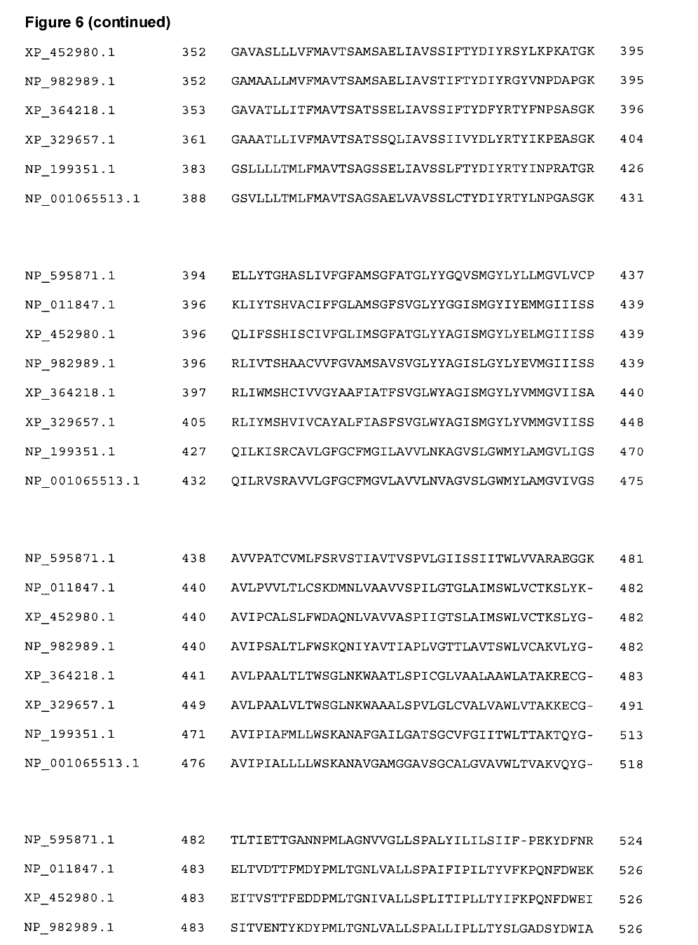

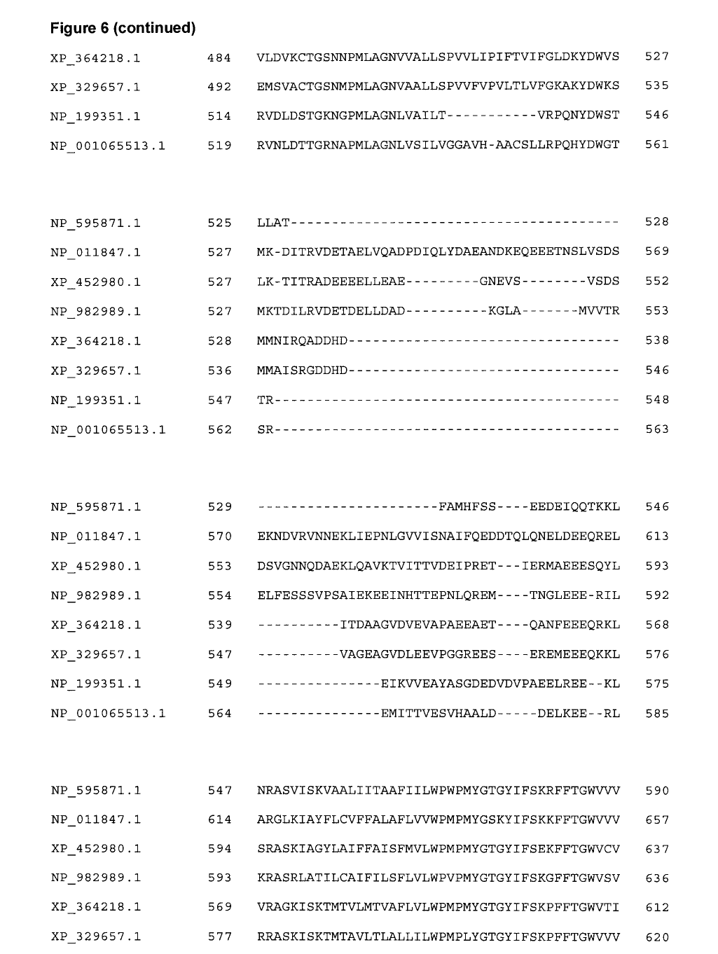

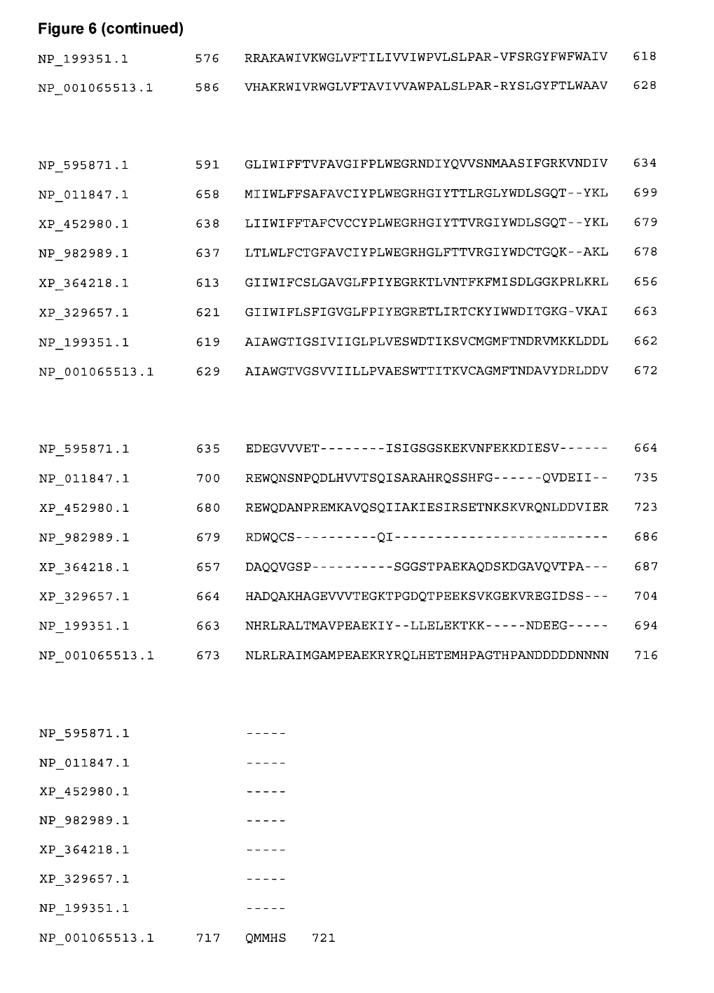

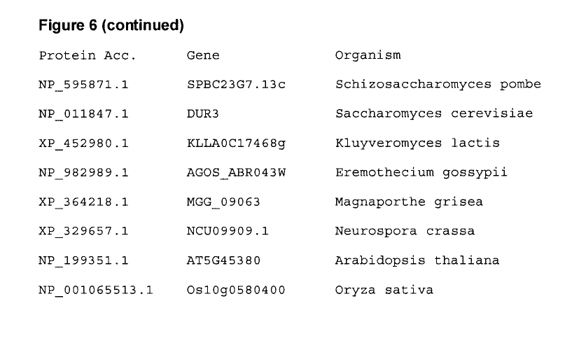

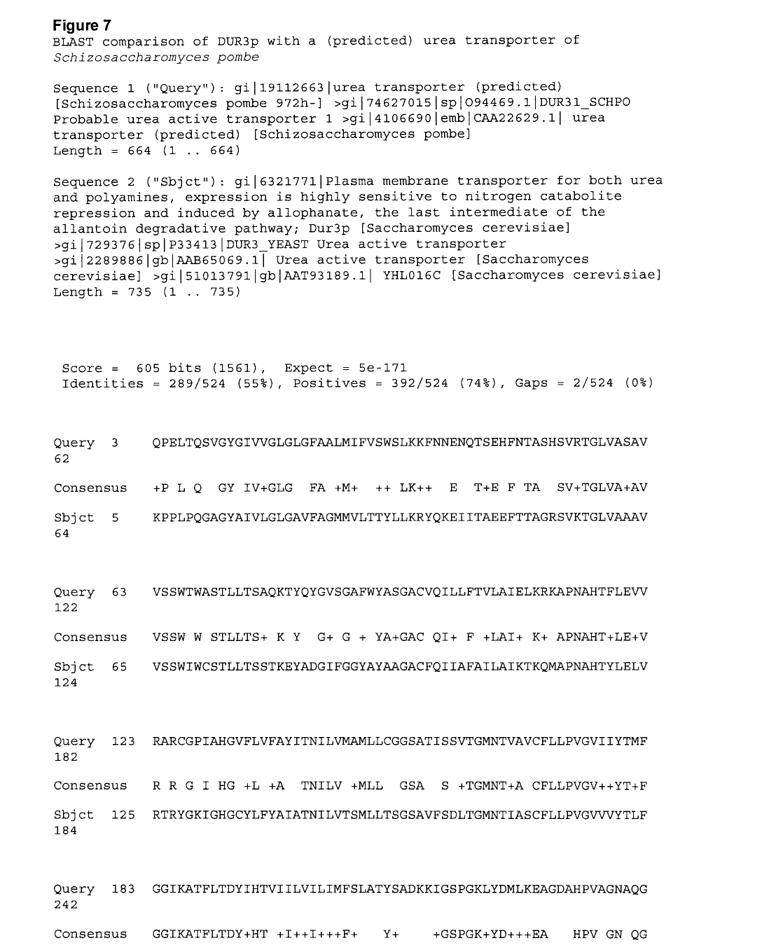

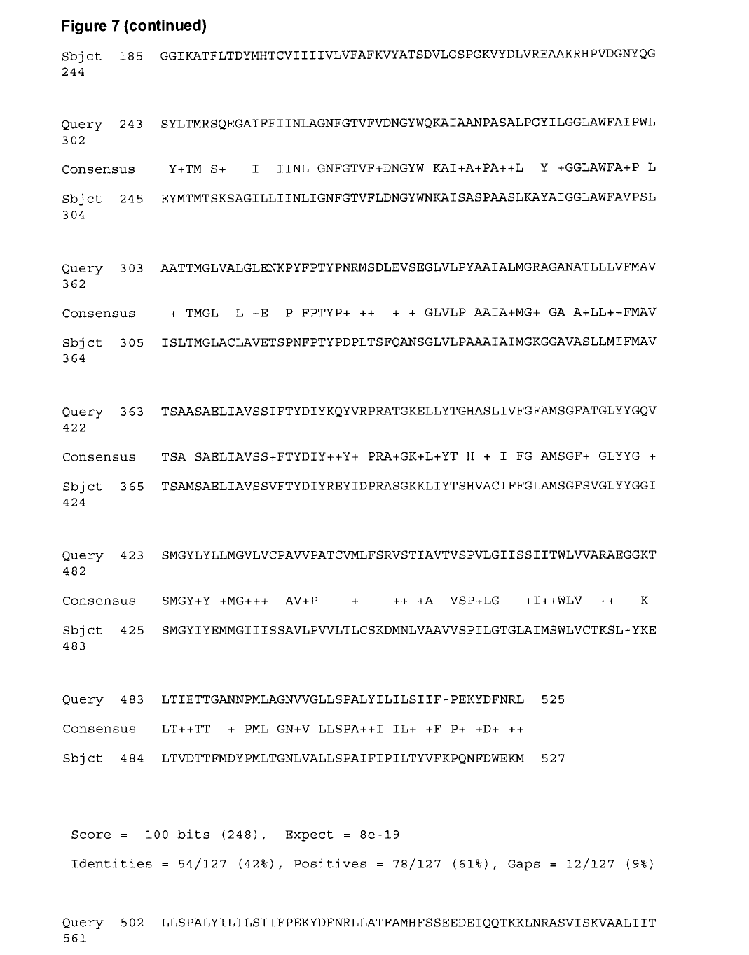

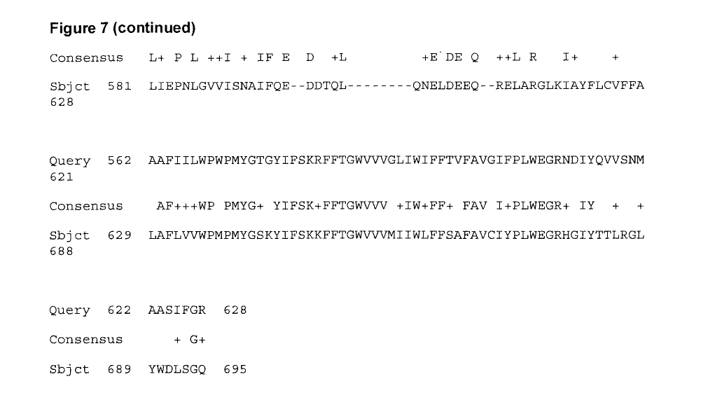

In alternative embodiments, site directed mutagenesis may be employed to alter the level of urea transport or urea degrading activity in a host. For example, site directed mutagenesis may be employed to remove NCR mediating elements from a yeast promoter, such as the DUR3 or DUR1,2 promoter. For example, the GATAA(G) boxes in the native DUR1,2 promoter sequence, as shown in FIG. 4, may be deleted or modified by substitution. In one embodiment, for example, one or both of the GATAA boxes may be modified by substituting a T for the G, so that the sequence becomes TATAA. Methods of site directed mutagenesis are for example disclosed in: Rothstein, 1991; Simon and Moore, 1987; Winzeler et al., 1999; and, Negritto et al., 1997. In alternative embodiments, the genes encoding for Gln3p and Gat1p that mediate NCR in S. cerevisiae may also be mutated to modulate NCR. Selected or engineered promoters lacking NCR may then be operatively linked to the DUR3 coding sequence, to mediate expression of DUR3 under fermenting conditions.

The relative urea transport or degrading enzymatic activity of a yeast strain of the invention may be measured relative to an untransformed parent strain. For example, transformed yeast strains of the invention may be selected to have greater urea transport or degrading activity than a parent strain under fermenting conditions, or an activity that is some greater proportion of the parent strain activity under the same fermenting conditions, such as at least 150%, 200%, 250%, 300%, 400% or 500% of the parent strain activity. Similarly, the activity of enzymes expressed or encoded by recombinant nucleic acids of the invention may be determined relative to the non-recombinant sequences from which they are derived, using similar multiples of activity.

In one aspect of the invention, a vector may be provided comprising a recombinant nucleic acid molecule having the DUR3 coding sequence, or homologues thereof, under the control of a heterologous promoter sequence that mediates regulated expression of the DUR3 polypeptide. To provide such vectors, the DUR3 open reading frame (ORF) from S. cerevisiae may be inserted into a plasmid containing an expression cassette that will regulate expression of the recombinant DUR3 gene. The recombinant molecule may be introduced into a selected yeast strain to provide a transformed strain having altered urea transport activity. In alternative embodiments, expression of a native DUR3 coding sequence homologue in a host such as S. cerevisiae may also be effected by replacing the native promoter with another promoter. Additional regulatory elements may also be used to construct recombinant expression cassettes utilizing an endogenous coding sequence. Recombinant genes or expression cassettes may be integrated into the chromosomal DNA of a host such as S. cerevisiae.

Promoters for use in alternative aspects of the invention may be selected from suitable native S. cerevisiae promoters, such as the PGK1 or CAR1 promoters. Such promoters may be used with additional regulator elements, such as the PGK1 and CAR1. terminators. A variety of native or recombinant promoters may be used, where the promoters are selected or constructed to mediate expression of urea degrading activities, such as DUR1,2p activities, under selected conditions, such as wine making conditions. A variety of constitutive promoters may for example be operatively linked to the DUR3 coding sequence.

According to one aspect of the invention, a method of fermenting a carbohydrate is provided, such as a method of fermenting wine, using a host, such as a yeast strain, transformed with a recombinant nucleic acid that modulates the urea transport (uptake) activity of the host. For example, the NCR of the DUR3 gene may be modulated to enhance the uptake of urea in a wine making yeast strain. In accordance with this aspect of the invention, fermentation of a grape must with the yeast strain may be carried out so as to result in the production of limited amounts of ethyl carbamate.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the specification, the word “comprising” is used as an open-ended term, substantially equivalent to the phrase “including, but not limited to”, and the word “comprises” has a corresponding meaning. Citation of references herein shall not be construed as an admission that such references are prior art to the present invention. All publications, including but not limited to patents and patent applications, cited in this specification are incorporated herein by reference as if each individual publication were specifically and individually indicated to be incorporated by reference herein and as though fully set forth herein. The invention includes all embodiments and variations substantially as hereinbefore described and with reference to the examples and drawings.

In one embodiment of the invention there is provided a yeast strain transformed to reduce nitrogen catabolite repression of urea transporter expression under fermenting conditions. For example, the urea transporter may be encoded by DUR3. and the urea transporter may be DUR3p.

In a further embodiment of the invention there is provided a yeast strain transformed to reduce nitrogen catabolite repression of both urea transporter expression and urea degradation enzyme expression under fermenting conditions. The urea transporter may be encoded by DUR3 and said urea degrading enzyme may be encoded by DUR1,2. and the urea transporter may be DUR3p and said urea degrading enzyme may be urea carboxylase/allophanate hydrolase.

In a further embodiment of the invention there is provided a yeast strain which has been transformed to continually uptake urea under fermenting conditions. The yeast may for example constitutively express DUR3.

In a further embodiment of the invention there is provided a yeast strain which has been transformed to continually uptake urea and also degrade urea under fermenting conditions. Wherein said yeast strain may constitutively express both DUR1,2 and DUR3.

In a further embodiment of the invention there is provided a method for modifying a yeast strain comprising transforming said yeast strain to reduce nitrogen catabolite repression of urea transporter expression under fermenting conditions. The urea transporter may for example be encoded by DUR3, and the urea transporter may be DUR3p.

In a further embodiment of the invention there is provided a method for modifying a yeast strain comprising transforming said yeast strain to reduce nitrogen catabolite repression of urea transporter expression and of urea degradation enzyme expression under fermenting conditions. The urea transporter may be encoded by DUR3 and said urea degrading enzyme may be encoded by DUR1,2. and the urea transporter may be DUR3p and said urea degrading enzyme may be urea carboxylase or allophanate hydrolase.

In a further embodiment of the invention there is provided a method for modifying a yeast strain to constitutively express DUR3. The method may include integration of the 1/2TRP1-PGKₚ-DUR3-PGKₜ-kanMX-1/2TRP1 cassette into the TRP1 locus. Alternatively, the method may include transforming said yeast strain with a recombinant nucleic acid comprising a coding sequence encoding DUR3p. Alternatively, the method may include transforming said yeast with a recombinant nucleic acid comprising a promoter not subject to nitrogen catabolite repression.

In a further embodiment of the invention there is provided a method of making a fermented beverage or food product using a yeast strain transformed to reduce nitrogen catabolite repression of urea transporter expression under fermenting conditions. The urea transporter may be encoded by DUR3, and the urea transporter may be DUR3p.

In a further embodiment of the invention there is provided a method of making a fermented beverage or food product using a yeast strain transformed to reduce nitrogen catabolite repression of both urea transporter expression and urea degradation enzyme expression under fermenting conditions. The urea transporter may be encoded by DUR3 and said urea degrading enzyme may be encoded by DUR1,2. The urea transporter may be DUR3p and said urea degrading enzyme may be urea carboxylase or allophanate hydrolase.

In a further embodiment of the invention there is provided the use of a transformed yeast strain that constitutively expresses DUR3 to reduce the concentration of ethyl carbamate in a fermented beverage or food product.

In a further embodiment of the invention there is provided the use of a transformed yeast strain that constitutively expresses both DUR1,2 and DUR3 to reduce the concentration of ethyl carbamate in a fermented beverage or food product.

In a further embodiment of the invention there is provided the use of a transformed yeast strain that constitutively expresses DUR3 to produce a wine having an ethyl carbamate concentration of less than 30 ppb.

In a further embodiment of the invention there is provided the use of a transformed yeast strain that constitutively expresses both DUR1,2 and DUR3 to produce a wine having an ethyl carbamate concentration of less than 30 ppb.

In a further embodiment of the invention there is provided a fermented beverage or food product having a reduced ethyl carbamate concentration produced using a transformed yeast strain that constitutively expresses DUR3.

In a further embodiment of the invention there is provided a fermented beverage or food product having a reduced ethyl carbamate concentration produced using a transformed yeast strain that constitutively expresses both DUR1,2 and DUR3.

In a further embodiment of the invention there is provided a wine having an ethyl carbamate concentration of less than 30 ppb produced using a transformed yeast strain that constitutively expresses DUR3.

In a further embodiment of the invention there is provided a wine having an ethyl carbamate concentration of less than 30 ppb produced using a transformed yeast strain that constitutively expresses both DUR1,2 and DUR3.

### EXAMPLES

The invention is herein further described with reference to the following, non-limiting, examples. A description of the experimental procedures employed follows the examples.

### Example 1

### Cloning and Constitutive Expression of the DUR3 Gene in Wine Strains of Saccharomyces cerevisiae

For clone selection the antibiotic resistance marker kanMX was used. Yeast strains UC Davis 522 (Montrachet), Prise de Mousse (EC1118), and K7-01 (sake yeast) have been transformed to constitutively express DUR3 alone or both DUR1,2 and DUR3. Extensive testing has indicated that the transformed yeast are substantially equivalent to their parental strains. That is, the only genetic and metabolic modifications are the intended constitutive expression of DUR3 or both DUR1,2 and DUR3.

### Example 2

### Transformation of Yeast with the DUR3 Gene Cassette

Yeast were transformed with recombinant nucleic acid containing the DUR3 gene under control of the PGK1 promoter and terminator signal. The PGK1 promoter is not subject to NCR. The DUR3 gene cassette—1/2TRP1-PGKₚ-DUR3-PGKₜkanMX-1/2TRP1.

### Example 3

### Fermentation Studies with the Recombinant Yeast to Establish the Occurrence of Reduced Ethyl Carbamate

Constitutive expression of DUR3 creates yeast strains which reabsorb urea that was excreted as a by-product of arginine metabolism, but they also absorb urea that is naturally present in the grape must. A significant reduction in ethyl carbamate is seen in wine exposed to the 522^{DUR3} yeast strain (˜81%), and a reduction of 25 and 13% is seen after exposure to the K7^{DUR3} and PDM^{DUR3} yeast strains, respectively (data in Table 1). It has also been shown that yeast that constitutively express DUR3 reduce ethyl carbamate concentrations as efficiently as yeast that constitutively express DUR1,2.

The combination of both DUR1,2 and DUR3 constitutive expression reduces ethyl carbamate to approximately the same extent as either DUR1,2 or DUR3 alone in the 552 and K7 yeast strains. The PDM^{EC-DUR3} (DUR1,2 and DUR3), however, is an example of a yeast strain that is able to reduce ethyl carbamate in wine must to a greater extent than either the PDM^{DUR3} (DUR3) or PDM^{EC} (DUR1,2) strains alone.

### Example 4

### Self-Cloning Cassette Allowing Removal of Selectable Marker

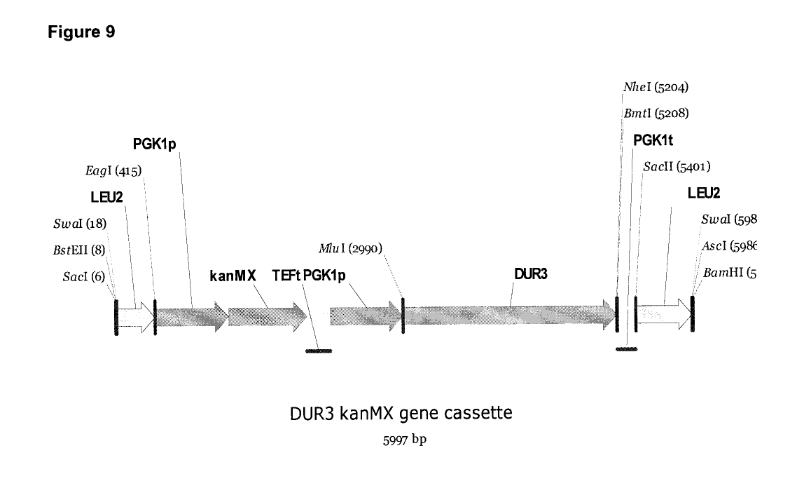

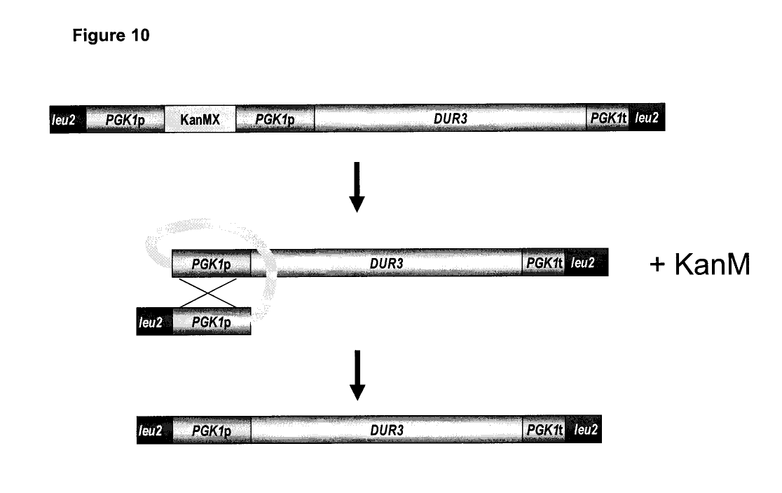

FIGS. 9 and 10 illustrate a DUR3 genetic cassette leu2-PGK1ₚ-kanMX-PGKₚ-DUR3-PGK1ₜleu2 allowing for selection of transformed yeast and subsequent removal of an antibiotic resistance marker via recombination of direct repeats, used in this example as described below.

Yeast were transformed with recombinant nucleic acid comprising the DUR3 gene under control of the PGK1 promoter and terminator signal that allows selection of transformed yeast and the subsequent removal of an antibiotic resistance marker via recombination of direct repeats. The PGK1 promoter is not subject to NCR. The DUR3 gene cassette—leu2-PGK1ₚ-kanMX-PGKₚ-DUR3-PGK1ₜ-leu2. Four strains were transformed with the leu2-PGK1ₚ-kanMX-PGKₚ-DUR3-PGK1ₜ-leu2 cassette: CY3079, Bordeaux Red, and DUR1,2-transformed strains D80ec- and D254ec-. This yielded 55 strains for D254ec-, 125 strains for D80ec-, approximately 200 strains for Bordeaux Red, and approximately 300 strains for CY3079. Approximately 20-60 clones per strain were chosen for mini-fermentations to determine EC reduction. Two CY3079 clones had EC reductions of 94.2% and 46.5% under laboratory conditions; three Bordeaux Red clones had EC reductions of 57.6%-64.9%; the D80ec- clones had EC reductions of 60.8%-66.1%; and two D254ec- clones had EC reductions of 87.5% and 75.1%.

### Experimental Procedures Employed for the Above Examples

Construction of pHVX2D3

In order to place DUR3 under the control of the constitutive PGK1 promoter and terminator signals, the DUR3 ORF was cloned into pHVX2. The DUR3 ORF was amplified from 522 genomic DNA using the following primers which contained Xho1 restriction enzyme sites built into their 5′ ends:

DUR3forXho1
(SEQ ID NO: 1)
(5′-AAAACTCGAGATGGGAGAATTTAAACCTCCGCTAC-3′)
DUR3revXho1
(SEQ ID NO: 2)
(5′-AAAACTCGAGCTAAATTATTTCATCAACTTGTCCGAAATGTG-3′).

Following PCR, 0.8% agarose gel visualization, and PCR cleanup (Qiagen, USA—PCR Purification Kit), both the PCR product (insert) and pHVX2 (vector) were digested with Xho1 (Roche, Germany). After the digested vector was treated with SAP (Fermentas, USA) to prevent recircularization, the insert and linearized-SAP treated vector were ligated overnight at 22° C. (T4 DNA Ligase—Fermentas, USA); the ligation mixture (5 μL) was used to transform DH5α™ competent cells (Invitrogen, USA) that were subsequently grown on 100 μg/mL Ampicillin (Fisher, USA) supplemented LB (Difco, USA) plates. Plasmids from a random selection of transformant colonies were harvested (Qiagen, USA—QIAprep Spin Miniprep kit) and digested by EcoR1 (Roche, Germany); PCR, using inside-outside primers, was done to identify plasmids with the desired insert. The resultant plasmid containing PGK1p-DUR3-PGK1t was named pHVX2D3.

Construction of pHVXKD3

The kanMX marker was obtained from pUG6 via double digestion with Xho1 and Sal1 (Fermentas, USA). Following digestion, the 1500 bp kanMX band was gel purified (Qiagen, USA—Gel Extraction Kit) and ligated into the Sal1 site of linearized-SAP treated pHVX2D3. The ligation mixture (5 μL) was used to transform DH5α™ competent cells which were grown on LB-Ampicillin (100 μg/mL). Recombinant plasmids were identified by HindIII (Roche, Germany) digestion of plasmids isolated from 24 randomly chosen colonies. The resultant plasmid containing PGK1p-DUR3-PGK1t-kanMX was named pHVXKD3

Construction of pUCTRP1

The TRP1 coding region was PCR amplified from 522 genomic DNA using TRP1 specific primers, each containing BamH1 and then Apa1 sites at their 5′ ends:

BamH1Apa1TRP1ORFfwd
(SEQ ID NO: 3)
(5′-AAAAAAGGATCCAAAAAAGGGCCCATGTCTGTTATTAATTTCACA
GG-3′)
BamH1Apa1TRP1ORFrev
(SEQ ID NO: 4)
(5′-AAAAAAGGATCCAAAAAAGGGCCCCTATTTCTTAGCATTTTTGAC
G-3′).

Following amplification, cleanup, and quantification, the ˜750 by fragment was ligated into the BamH1 (Roche, Germany) site of linearized-SAP treated pUC18. Recombinant plasmids were identified primarily through blue/white screening (growth on LB-Ampicillin supplemented with 50 μg/mL Xgal) and subsequently confirmed through HindIII/EcoR1 digestion. The resultant plasmid containing TRP1 was called pUCTRP1.

Construction of pUCMD

The PGK1p-DUR3-PGK1t-kanMX cassette located within pHVXKD3 was amplified from pHVXKD3 plasmid DNA using cassette specific primers:

pHVXKfwdlong
(SEQ ID NO: 5)
(5′-CTGGCACGACAGGTTTCCCGACTGGAAAGCGGGCAGTGAG-3′)
pHVXKrevlong
(SEQ ID NO: 6)
(5′-CTGGCGAAAGGGGGATGTGCTGCAAGGCGATTAAGTTGGG-3′).

Following amplification, cleanup, and quantification, the ˜6500 bp blunt end PCR generated fragment was treated with polynucleotide kinase (New England Biolabs, USA) in order to facilitate ligation into the blunt EcoRV (Fermentas, USA) site of linearized-SAP treated pUCTRP1.

Recombinant plasmids were initially identified using E-lyse analysis and later confirmed via Apa/(Stratagene, USA)/Sal1 digestion. Briefly, E-lyse efficiently screens large numbers of colonies for the presence of plasmid DNA by lysing the colonies within the wells of an agarose gel, followed by electrophoresis. More specifically, after patching onto selective media, small aliquots of colonies were suspended in 5 μL TBE buffer and then mixed with 10 μL SRL buffer (25% v/v sucrose, 50 μg/mL RNase, 1 mg/mL lysozyme). After mixing by pipetting, cell suspensions were loaded into the wells of a 0.2% (w/v) SDS—0.8% (w/v) agarose gel. After the cell suspension in the wells had become clear indicating cell lysis (˜30 min), the DNA was electrophoresed at 20 V for 45 min, then at 80 V for 45 min. Finally the gel was stained as required with SYBR™ Safe (Invitrogen, USA).

Transformation of the Linear DUR3 Cassette into S. cerevisiae and Selection of Transformants

The 6536 bp DUR3 cassette was cut from pUCMD using Apa1 (Stratagene, USA) and visualized on a 0.8% agarose gel. From the gel, the expected 6536 bp band was resolved and extracted (Qiagen, USA—Gel extraction kit). After extraction, clean up, and quantification using a Nanodrop ND-1000 spectrophotometer (Nanodrop, USA), 250 ng of linear cassette was used to transform S. cerevisiae strains 522, 522^{Ec-}, PDM, PDM^{Ec-}, K7 and K7^{Ec-}. Yeast strains were transformed using the lithium acetate/polyethylene glycol/ssDNA method. Following transformation, cells were left to recover in YPD at 30° C. for 2 hours before plating on to YPD plates supplemented with 300 μg/mL G418 (Sigma, USA). Plates were incubated at 30° C. until colonies appeared.

Calona Chardonnay

Unfiltered Chardonnay grape juice (23.75 Brix, pH 3.41, ammonia 91.6 mg/L, FAN 309.6 mg/L) was obtained from Calona Vineyards, Okanagan Valley and used for the inoculation of the modified yeast. Single colonies of parental strains (522, PDM, K7, and K9) as well as appropriate functionally enhanced strains from freshly streaked YPD plates, were inoculated into 5 mL YPD and grown overnight (30° C.—rotary wheel). Cells were subcultured into 50 mL YPD (OD₆₀₀=0.05) and again grown overnight (30° C.—180 rpm shaker bath). Cells were harvested by centrifugation (5000 rpm, 4° C., 5 min) and washed once with 50 mL sterile water. Cell pellets were resuspended in 5 mL sterile water and OD₆₀₀ measured. Cell suspensions were used to inoculate sterile 250 mL Schott bottles filled with 200 mL unfiltered Chardonnay juice obtained from Calona Vineyards, Kelowna, BC, Canada to a final OD₆₀₀=0.1. Bottles were aseptically sealed with sterilized (70% v/v ethanol) vapour locks filled with sterile water. Sealed bottles were incubated at 20° C., and weighed daily to monitor CO₂ production. Data were plotted in Excel to generate fermentation profiles. At the end of fermentation, cells were removed by centrifugation (5000 rpm, 4° C., 5 min), and ˜50 mL of wine was decanted into sterile 50 mL Schott bottles. Bottles were incubated in a 70° C. water bath for exactly 48 hours to maximize EC production, and then stored at 4° C. until GC/MS analysis.

Quantification of Ethyl Carbamate in Wine by SPME and GC-MS

Chardonnay wine was heated at 70° C. for 48 hr to stimulate ethyl carbamate production. A 10-mL wine sample was pipetted into a 20-mL sample vial. A small magnetic stirring bar and 3 g of NaCl were added and the vial was capped with PTFE/silicone septum. The vial was placed on a stirrer at 22° C. and allowed to equilibrate, with stirring, for 15 min. A SPME fiber (65 μm carbowax/divinylbenzene) was conditioned at 250° C. for 30 min before use. After sample equilibration, the fiber was inserted into the headspace. After 30 min, the fiber was removed from the sample vial and inserted into the injection port for 15 min. A blank run was performed before each sample run. Quantification was done using an external standard method. An ethyl carbamate (Sigma-Aldrich, Milwaukee, Wis.) standard stock solution was prepared at 0.1 mg/mL in distilled H2O containing 12% (v/v) ethanol and 1 mM tartaric acid at pH 3.1. Calibration standards were prepared with EC concentrations of 5, 10, 20, 40, 90 μg/L. The standard solutions were stored in the refrigerator at 4° C. Ethyl carbamate in wine was quantified using an Agilent 6890N GC interfaced to a 5973N Mass Selective Detector. A 60 m×0.25 mm i.d., 0.25 μm thickness DBWAX fused-silica open tubular column (J&W Scientific, Folsom, Calif.) was employed. The carrier gas was ultra-high-purity helium at a constant flow of 36 cm/s. The injector and transfer line temperature was set at 250° C. The oven temperature was initially set at 70° C. for 2 min, then raised to 180° C. at 8° C./min and held for 3 min. The temperature was then programmed to increase by 20° C./min to 220° C. where it was held for 15 min. The total run time was 35.75 min. The injection mode was splitless for 5 min (purge flow: 5 mL/min, purge time: 5 min). The MS was operated in selected ion monitoring (SIM) mode with electron impact ionization; MS quad temperature 150° C. and MS source temperature 230° C. The solvent delay was 8 min. Specific ions 44, 62, 74, 89 were monitored with a dwell time of 100 msec. Mass 62 was used for quantification against the mass spectrum of the authentic EC standard.

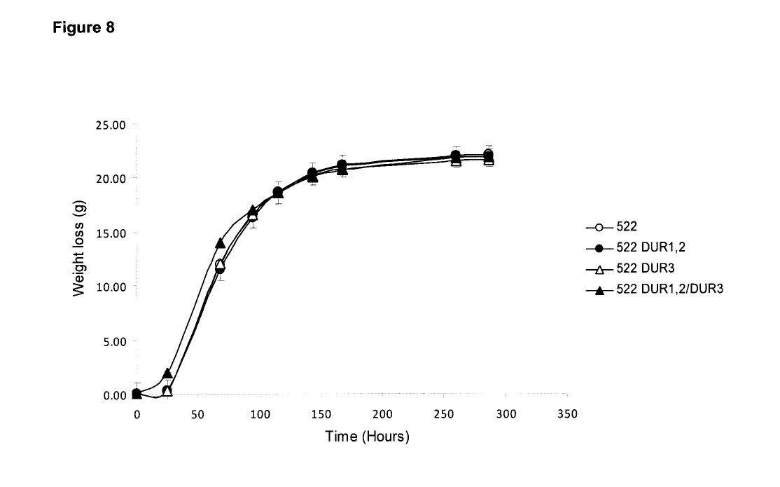

Fermentation profiles are presented in FIG. 8 and the final amount of ethanol produced by the functionally enhanced and control strains are shown in Table 2.

TABLE 1
Summary of ethyl carbamate reduction during wine making. Ethyl
carbamate (μg/L) in Chardonnay wine produced by Sake yeast strains
(K7, K7^{EC-} (8),^{,} K7^{DUR3}, K7^{EC-DUR3}) and wine yeast strains (522,
522^{EC-,}, 522^{DUR3}, 522^{EC-DUR3}, PDM, PDM^{EC-,}, PDM^{DUR3}, PDM^{EC-DUR3})
from unfiltered Calona Chardonnay must was quantified by GC/MS.
Fermentations were incubated to completion (~300 hrs) at 20° C.

| | | K7^{EC-} | | |
|---|---|---|---|---|
| | K7 | (#8) | K7^{DUR3} | K7^{EC-DUR3} |
| Replicate 1 | 33.55 | 36.66 | 30.80 | 36.20 |
| Replicate 2 | 42.82 | 32.21 | 32.03 | 37.39 |
| Replicate 3 | 41.04 | 36.64 | 25.26 | 25.85 |
| Average (n = 3) | 39.14 | 35.17 | 29.36 | 33.15 |
| STDEV | 4.92 | 2.56 | 3.61 | 6.35 |
| % Reduction | — | 10.14 | 24.97 | 15.31 |
| | 522 | 522^{EC-} | 522^{DUR3} | 522^{EC-DUR3} |
| Replicate 1 | 210.25 | 34.15 | 32.60 | 41.50 |
| Replicate 2 | 193.40 | 44.16 | 34.03 | 42.35 |
| Replicate 3 | 184.27 | 36.54 | 42.82 | 29.80 |
| Average (n = 3) | 195.97 | 38.28 | 36.48 | 37.88 |
| STDEV | 13.18 | 5.23 | 5.53 | 7.01 |
| % Reduction | — | 80.47 | 81.38 | 80.67 |
| | PDM | PDM^{EC-} | PDM^{DUR3} | PDM^{EC-DUR3} |
| Replicate 1 | 44.73 | 28.86 | 39.01 | 24.34 |
| Replicate 2 | 45.93 | 34.50 | 43.03 | 24.08 |
| Replicate 3 | 48.07 | 33.61 | 40.81 | 25.17 |
| Average (n = 3) | 46.24 | 32.32 | 40.95 | 24.53 |
| STDEV | 1.69 | 3.03 | 2.01 | 0.57 |
| % Reduction | — | 31.06 | 12.66 | 47.68 |

^{EC-}constitutive expression of DUR1, 2
^{DUR3}constitutive expression of DUR3
^{EC-DUR3}combined constitutive expression of DUR1, 2 and DUR3

TABLE 2
Ethanol produced by wine yeast strains (522, 522^{DUR1, 2} [an
alternative designation for 522^{EC-}] 522^{DUR3}, and 522^{DUR1, 2/DUR3}
[an alternative designation for 522^{EC-DUR3}]) in Chardonnay
wine. Ethanol content (% v/v) was measured by LC at the end
of fermentation. Fermentation profiles are given in FIGS.
3. Data were analyzed for statistical significance (p ≦
0.05) using two factor ANOVA analysis.

| | 522 | 522^{DUR1, 2} | 522^{DUR3} | 522^{DUR1, 2/DUR3} |
|---|---|---|---|---|
| Replicate 1 | 13.65 | 13.71 | 13.74 | 13.54 |
| Replicate 2 | 13.60 | 13.65 | 13.71 | 13.62 |
| Replicate 3 | 13.71 | 13.66 | 13.55 | 13.58 |
| Ethanol average | 13.65 | 13.67n | 13.67n | 13.58n |
| (n = 3) | | | | |
| STDEV | 0.06 | 0.03 | 0.10 | 0.04 |

s: significant at p ≦ 0.05, n: non-significant

1. A yeast strain transformed to reduce nitrogen catabolite repression of gene expression of a urea transporter protein under fermenting conditions.

2. The yeast strain of claim 1, wherein the urea transporter is encoded by DUR3.

3. The yeast strain of claim 1, wherein the urea transporter is DUR3p.

4. The yeast strain of claim 3, further transformed to reduce nitrogen catabolite repression of gene expression of a urea degradation enzyme under the fermenting conditions.

5. The yeast strain of claim 4, wherein the urea degrading enzyme is encoded by DUR1,2.

6. The yeast strain of claim 4, wherein the urea degrading enzyme is urea carboxylase/allophanate hydrolase or urea amidolyase.

7. The yeast strain of claim 1, transformed to continually uptake urea under fermenting conditions.

8. The yeast strain of claim 7, wherein the yeast has been transformed to constitutively express DUR3.

9. The yeast strain of claim 4, transformed to continually uptake urea and continually degrade urea under the fermenting conditions.

10. The yeast strain of claim 9, wherein the yeast strain is transformed to constitutively express DUR1,2 and DUR3.

11. A method for modifying a yeast strain comprising transforming the yeast strain to reduce nitrogen catabolite repression of gene expression of a urea transporter under fermenting conditions.

12. The method of claim 11, wherein the urea transporter is encoded by DUR3.

13. The method of claim 11, wherein the urea transporter is DUR3p.

14. The method of claim 13, wherein the yeast strain is further transformed to reduce nitrogen catabolite repression of gene expression of a urea degradation enzyme under the fermenting conditions.

15. The method of claim 14, wherein the urea degrading enzyme is encoded by DUR1,2.

16. The method of claim 14, wherein the urea degrading enzyme is urea carboxylase, allophanate hydrolase, or urea amidolyase.

17. The method of claim 11, wherein the yeast strain is transformed to constitutively express DUR3.

18. The method of any one of claim 17, wherein the yeast strain is transformed with a recombinant nucleic acid comprising a coding sequence encoding DUR3p.

19. The method of claim 18, wherein the coding sequence encoding DUR3p is operatively linked to a promoter that is not subject to nitrogen catabolite repression.

20. A method of making a fermented beverage or food product, comprising maintaining the yeast strain of claim 1 under fermenting conditions, to produce a fermented beverage or food product having a reduced concentration of ethyl carbamate.

21. (canceled)

22. The method of claim 20, wherein the fermented beverage or food product has an ethyl carbamate concentration of less than 30 ppb.

23. (canceled)

24. The method of claim 20, wherein the fermented beverage or food product is a wine.

25. (canceled)

26. A fermented beverage or food product having a reduced ethyl carbamate concentration produced using the transformed yeast strain of claim 1.

27. The fermented beverage or food product of claim 26, which is a wine.

28. The wine of claim 27, having an ethyl carbamate concentration of less than 30 ppb.

29. The fermented beverage or food product of claim 26, comprising a yeast strain transformed to reduce nitrogen catabolite repression of gene expression of a urea transporter protein under fermenting conditions.


SEQUENCE LISTING (17 sequences)

SEQ ID NO: 1 - DNA, 35 bp, Artificial Sequence
  DUR3forXho1
aaaactcgagatgggagaatttaaacctccgctac

SEQ ID NO: 2 - DNA, 42 bp, Artificial Sequence
  DUR3revXho1
aaaactcgagctaaattatttcatcaacttgtccgaaatgtg

SEQ ID NO: 3 - DNA, 47 bp, Artificial Sequence
  BamH1Apa1TRP1ORFfwd
aaaaaaggatccaaaaaagggcccatgtctgttattaatttcacagg

SEQ ID NO: 4 - DNA, 46 bp, Artificial Sequence
  BamH1Apa1TRP1ORFrev
aaaaaaggatccaaaaaagggcccctatttcttagcatttttgacg

SEQ ID NO: 5 - DNA, 40 bp, Artificial Sequence
  pHVXKfwdlong
ctggcacgacaggtttcccgactggaaagcgggcagtgag

SEQ ID NO: 6 - DNA, 40 bp, Artificial Sequence
  pHVXKrevlong
ctggcgaaagggggatgtgctgcaaggcgattaagttggg

SEQ ID NO: 7 - protein, 735 aa, Saccharomyces cerevisiae
MGEFKPPLPQGAGYAIVLGLGAVFAGMMVLTTYLLKRYQKEIITAEEFTTAGRSVKTGLVAAAVVSSWIWCSTLLTSSTKEYADGIFGGYAYAAGACFQIIAFAILAIKTKQMAPNAHTYLELVRTRYGKIGHGCYLFYAIATNILVTSMLLTSGSAVFSDLTGMNTIASCFLLPVGVVVYTLFGGIKATFLTDYMHTCVIIIIVLVFAFKVYATSDVLGSPGKVYDLVREAAKRHPVDGNYQGEYMTMTSKSAGILLIINLIGNFGTVFLDNGYWNKAISASPAASLKAYAIGGLAWFAVPSLISLTMGLACLAVETSPNFPTYPDPLTSFQANSGLVLPAAAIAIMGKGGAVASLLMIFMAVTSAMSAELIAVSSVFTYDIYREYIDPRASGKKLIYTSHVACIFFGLAMSGFSVGLYYGGISMGYIYEMMGIIISSAVLPVVLTLCSKDMNLVAAVVSPILGTGLAIMSWLVCTKSLYKELTVDTTFMDYPMLTGNLVALLSPAIFIPILTYVFKPQNFDWEKMKDITRVDETAELVQADPDIQLYDAEANDKEQEEETNSLVSDSEKNDVRVNNEKLIEPNLGVVISNAIFQEDDTQLQNELDEEQRELARGLKIAYFLCVFFALAFLVVWPMPMYGSKYIFSKKFFTGWVVVMIIWLFFSAFAVCIYPLWEGRHGIYTTLRGLYWDLSGQTYKLREWQNSNPQDLHVVTSQISARAHRQSSHFGQVDEII

SEQ ID NO: 8 - DNA, 2208 bp, Saccharomyces cerevisiae
atgggagaatttaaacctccgctacctcaaggcgctgggtacgctattgtattgggcctaggggccgtatttgcaggaatgatggttttgaccacttatttactgaaacgttatcaaaaggaaatcatcacagcagaagaattcaccaccgccggtagatctgtaaaaaccggcttagtggctgcagccgtggtttctagttggatctggtgttctacattgttaacgtcgtcaacaaaggaatatgcagacggtatatttggcggttatgcgtacgctgctggcgcatgcttccaaattattgcattcgcaattttggcaattaaaaccaagcaaatggctcccaatgcgcacacatatttagaattagtgagaacaagatatggtaagatcggccatggttgctacttgttttatgccatcgcgacgaatattttagtcacttcaatgcttttaacttcaggttctgctgtctttagtgatttaaccgggatgaacactatcgcatcatgttttttactgcctgtgggtgttgttgtttatactctatttggtgggattaaagcaactttcttaacggactatatgcacacatgtgtcattatcatcattgtcctcgtatttgcctttaaagtttatgcgactagtgatgttttaggctcaccgggaaaagtttatgacttagttcgtgaagccgccaagaggcatccagtagacggtaactatcagggtgaatatatgaccatgacatccaaatccgctggtattttattaattattaacctgattggaaatttcggcaccgttttccttgataatggttattggaataaagcgatttctgctagtcccgcagcgagtttgaaagcatatgccatcggtgggttagcatggtttgcagtaccttctttgatttcattgaccatgggattagcatgtcttgcggtggaaacgtctccgaacttccccacctatcctgatccacttacttcgttccaggcaaattctgggttagtcttgccggcagctgcaattgctatcatgggtaaggggggtgctgtggcatcgctgctaatgattttcatggccgtcacatctgctatgtctgctgaactaattgccgtttcatctgttttcacttacgatatctatagagaatatattgatcctcgtgcaagcggtaagaaattgatttacacatcacacgttgcttgtatcttttttggtcttgccatgagtggattttcggttggtttatactatggtggtatttctatgggttatatctatgaaatgatgggtataattattagtagtgcagtattacctgtcgttttgaccttatgttccaaagacatgaatttggtggccgctgtagtgtcgcctattttgggcacaggactggctataatgtcatggcttgtctgtaccaaatccctttataaagaattgaccgtggatactacgttcatggattatccaatgttaacaggtaacttggtggctttgctatcaccagccatttttattcctattttaacgtatgtgtttaagccacaaaattttgactgggagaaaatgaaagatattactagagttgacgaaactgcagagttagttcaggctgaccctgatatccagctttacgatgctgaagctaacgataaggaacaagaagaagaaacaaattctctggtctcagatagtgaaaaaaacgatgttagagtaaataatgaaaaattgattgagcctaaccttggtgttgtaataagtaatgccatttttcaagaagatgacacacagttacaaaatgaattagacgaagaacaaagagaactagcacgtggtttaaaaattgcatacttcctatgtgtttttttcgctttggcatttttggtagtttggcccatgcccatgtatggttccaaatatatcttcagtaaaaaattctttaccggttgggttgttgtgatgatcatctggctttttttcagtgcgtttgccgtttgtatttatccactctgggaaggtaggcatggtatatataccactttgcgaggcctttactgggatctatctggtcaaacttataaattaagggaatggcaaaattcgaacccacaagatctgcatgtagtaacaagccaaattagtgcgagagcacatagacaatcatcacatttcggacaagttgatgaaataatttag

SEQ ID NO: 9 - DNA, 375 bp, Saccharomyces cerevisiae
  misc_feature (1)..(375): Upstream region of the DUR1,2 gene ending at the DUR1,2 start codon ATG
tcccctatttcatagggcactctgttcgcagttagcgcaagccattgagataagacacgcagatgttttgctttttcctgcttcttaatttgaatcgagctttgttagacgttgttggaaattgaatgtttgtatttaaacactagagcagatgaggtgtgagatttgtatactcgctcacttctgaatatcaggctctcttagctaagctttttttttctaggatcatataggctcaagtttttataagcttatattaatatatcagtggagcagctgatatacaccaaatttcaatttacattaatataaaagataaaaaatagaaatatcttttttatagtcacaataaatttcagttttgattaaaaaatg

SEQ ID NO: 10 - DNA, 489 bp, Saccharomyces cerevisiae
  misc_feature (1)..(489): Portion of the upstream region of the DUR3 gene
ctcttaaagatgcaaaagcagtcatacaggtatttgttttatgcagcaaggctgactcaaactaggcattccaattctgtttatctattggtgccgcaccttttttccgatgatgaaatgaggcggcgcacatttgggtgttatcttatcgggatattttatgatagcaaatgcgacagacaataatgctcgagagtggacaggccaatagtcagttgcagcatagaaacaagcaggcgtacctatgggaaaggtgtaactcatcattgcgctttctacggtacggttatccttagataacaggcaagatgtgctctattcatcggtgttgcatgaagatacgtcttgttcgccaagtatataaaatgaagcagtagtatagccgtagctttactattgttagcagtttcccttgtctagattcttaaattgggttcatccaagaaaaagtaataacataaatttgtcattgttatagtatgggagaat

SEQ ID NO: 11 - protein, 664 aa, Schizosaccharomyces pombe
MVQPELTQSVGYGIVVGLGLGFAALMIFVSWSLKKFNNENQTSEHFNTASHSVRTGLVASAVVSSWTWASTLLTSAQKTYQYGVSGAFWYASGACVQILLFTVLAIELKRKAPNAHTFLEVVRARCGPIAHGVFLVFAYITNILVMAMLLCGGSATISSVTGMNTVAVCFLLPVGVIIYTMFGGIKATFLTDYIHTVIILVILIMFSLATYSADKKIGSPGKLYDMLKEAGDAHPVAGNAQGSYLTMRSQEGAIFFIINLAGNFGTVFVDNGYWQKAIAANPASALPGYILGGLAWFAIPWLAATTMGLVALGLENKPYFPTYPNRMSDLEVSEGLVLPYAAIALMGRAGANATLLLVFMAVTSAASAELIAVSSIFTYDIYKQYVRPRATGKELLYTGHASLIVFGFAMSGFATGLYYGQVSMGYLYLLMGVLVCPAVVPATCVMLFSRVSTIAVTVSPVLGIISSIITWLVVARAEGGKTLTIETTGANNPMLAGNVVGLLSPALYILILSIIFPEKYDFNRLLATFAMHFSSEEDEIQQTKKLNRASVISKVAALIITAAFIILWPWPMYGTGYIFSKRFFTGWVVVGLIWIFFTVFAVGIFPLWEGRNDIYQVVSNMAASIFGRKVNDIVEDEGVVVETISIGSGSKEKVNFEKKDIESV

SEQ ID NO: 12 - protein, 723 aa, Kluyveromyces lactis
MAVMEAPLPQGAGYAVVVGLGFVFAFAMILTTYVLRRYQKEIITAEEFATAGRSVKTGLIAAAVVSSWTWAATLLQSTTMVYKVGISGGYFYAAGAGYQVILFSALAIKCKQRAPNAHTYLEIIKARYGTIGHFCYMFYALATNVLVTAMLLTGGSAVVSDLTGMHTVAACFLLPVGVVLYTIFGGIKATFLTDYVHTIVIIVIIMIFAFTVYATNSQLGSPKAVYDLVREAAKAHPIEGNAGGEYLTMRSRSGGIFFVINIVGNFGTVFLDAGYWNKAISSSPAAALPGYVLGGLSWIAIPNLISLAMGLACVALESSPNFPTYPERLTAEQVSAGLVLPTAAVTLLGKGGAVASLLLVFMAVTSAMSAELIAVSSIFTYDIYRSYLKPKATGKQLIFSSHISCIVFGLIMSGFATGLYYAGISMGYLYELMGIIISSAVIPCALSLFWDAQNLVAVVASPIIGTSLAIMSWLVCTKSLYGEITVSTTFEDDPMLTGNIVALLSPLITIPLLTYIFKPQNFDWEILKTITRADEEEELLEAEGNEVSVSDSDSVGNNQDAEKLQAVKTVITTVDEIPRETIERMAEEESQYLSRASKIAGYLAIFFAISFMVLWPMPMYGTGYIFSEKFFTGWVCVLIIWIFFTAFCVCCYPLWEGRHGIYTTVRGIYWDLSGQTYKLREWQDANPREMKAVQSQIIAKIESIRSETNKSKVRQNLDDVIER

SEQ ID NO: 13 - protein, 686 aa, Eremothecium gossypii
MDHLNPPLSQGVGYAIVVGLGAVFAIGMVMTTYIFERYQREVITAEEFATARRTVKTGLIASAVVSSWTWQPRYCQSTTMAYKVGVSGPFYYAAGACVQIILFSTLAIKCKQRAPNAHTFLEIIKARYGRKAHILHMCYALVTNVLVTTMLLTGGSAVVSELTGMHTAAACFLLPVGVIIYTLFGGIKATFLTDYVHTVIIVGIILTFTFSVYRTSDMLGSASKVYDLLREAARQHPVKGNRNGEYLTMKSESGGIFFVVSLVGNFGTVLLDNGYFTKAFSSSPAAALPGYVVGGIVWFAIPCLVATSLGLACLALELLPSFPNYPSRLSQEQVDAGLVLPVAAFNLLGKGGAMAALLMVFMAVTSAMSAELIAVSTIFTYDIYRGYVNPDAPGKRLIVTSHAACVVFGVAMSAVSVGLYYAGISLGYLYEVMGIIISSAVIPSALTLFWSKQNIYAVTIAPLVGTTLAVTSWLVCAKVLYGSITVENTYKDYPMLTGNLVALLSPALLIPLLTYSLGADSYDWIAMKTDILRVDETDELLDADKGLAMVVTRELFESSSVPSAIEKEEINHTTEPNLQREMTNGLEEERILKRASRLATILCAIFILSFLVLWPVPMYGTGYIFSKGFFTGWVSVLTLWLFCTGFAVCIYPLWEGRHGLFTTVRGIYWDCTGQKAKLRDWQCSQI

SEQ ID NO: 14 - protein, 687 aa, Magnaporthe grisea
MSTHVPAALDQGTGYGIVLGLGALFAFGMIFVTFVLKRYNAERQTSEMFNTAGRTVKSGLVGSAVVSSWTWAATLLQSTGVCYRYGVSGPFWYASGATVQIILFATLAIELKRRAPNAHTFLEVIKARFGTAAHITFMVFGLVTNILVSLMLIVGGSATVNALTGMHTIAAIYLLPVGVVAYTMVGGLKATILTDWVHTFILLVIIIIFALTTYATSEDLGSPSAVYDLLVEAASRHPVEGNKDGSYLTMQSKEGAIFFVINIVGNFGTVFLDNGYYNKAIAASPVHALPGYILGGLSWFAIPWLTATTMGLAAIALESNPRFPTFPNRMSDDEVSEGLVLPYAAVALMGKGGAVATLLITFMAVTSATSSELIAVSSIFTYDFYRTYFNPSASGKRLIWMSHCIVVGYAAFIATFSVGLWYAGISMGYLYVMMGVIISAAVLPAALTLTWSGLNKWAATLSPICGLVAALAAWLATAKRECGVLDVKCTGSNNPMLAGNVVALLSPVVLIPIFTVIFGLDKYDWVSMMNIRQADDHDITDAAGVDVEVAPAEEAETQANFEEEQRKLVRAGKISKTMTVLMTVAFLVLWPMPMYGTGYIFSKPFFTGWVTIGIIWIFCSLGAVGLFPIYEGRKTLVNTFKFMISDLGGKPRLKRLDAQQVGSPSGGSTPAEKAQDSKDGAVQVTPA

SEQ ID NO: 15 - protein, 704 aa, Neurospora crassa
MAGGADTTVKIETATALNQGVGYGIVIGLGALFALGMIFVTFILKRYNRELQTSEMFNTAGRTVKSGLVGSAVVSSWTWAATLLQSSGVCYRYGVSGPLWYASGATVQILLFATLAIELKRRAPNAHTYLEVIRARFGTLPHIVFMIFGLMTNILVSLMLIVGGSATINALTGMHTIAAIYLLPVGVVAYTLVGGLKATILTDWIHTFILLIIIIVFALSAYASSEVLGSPSAVYDLLVKAAAAHPVDGNHEGSYLTMRSREGAIFFVINIVGNFGTVFLDNGYYNKAIAASPVHALPGYILGGLCWFAIPWLTATTMGLSGLALESSPRFPTYPNRMPEADVSAGLVLPYAAVALLGKGGAAATLLIVFMAVTSATSSQLIAVSSIIVYDLYRTYIKPEASGKRLIYMSHVIVCAYALFIASFSVGLWYAGISMGYLYVMMGVIISSAVLPAALVLTWSGLNKWAAALSPVLGLCVALVAWLVTAKKECGEMSVACTGSNMPMLAGNVAALLSPVVFVPVLTLVFGKAKYDWKSMMAISRGDDHDVAGEAGVDLEEVPGGREESEREMEEEQKKLRRASKISKTMTAVLTLALLILWPMPLYGTGYIFSKPFFTGWVVVGIIWIFLSFIGVGLFPIYEGRETLIRTCKYIWWDITGKGVKAIHADQAKHAGEVVVTEGKTPGDQTPEEKSVKGEKVREGIDSS

SEQ ID NO: 16 - protein, 694 aa, Arabidopsis thaliana
MATCPPFDFSTKYYDGDGGCQRQSSFFGGTTVLDQGVGYAVILGFGAFFAVFTSFLVWLEKRYVGARHTSEWFNTAGRNVKTGLIASVIVSQWTWAATILQSSNVAWQYGVSGPFWYASGATIQVLLFGVMAIEIKRKAPNAHTVCEIVKARWGTATHIVFLVFCLATNVVVTAMLLLGGSAVVNALTGVNLYAASFLIPLGVVVYTLAGGLKATFLASYVHSVIVHVALVVFVFLVYTSSKELGSPSVVYDRLKDMVAKSRSCTEPLSHHGQACGPVDGNFRGSYLTMLSSGGAVFGLINIVGNFGTVFVDNGYWVSAIAARPSSTHKGYLLGGLVWFAVPFSLATSLGLGALALDLPISKDEADRGLVPPATAIALMGKSGSLLLLTMLFMAVTSAGSSELIAVSSLFTYDIYRTYINPRATGRQILKISRCAVLGFGCFMGILAVVLNKAGVSLGWMYLAMGVLIGSAVIPIAFMLLWSKANAFGAILGATSGCVFGIITWLTTAKTQYGRVDLDSTGKNGPMLAGNLVAILTVRPQNYDWSTTREIKVVEAYASGDEDVDVPAEELREEKLRRAKAWIVKWGLVFTILIVVIWPVLSLPARVFSRGYFWFWAIVAIAWGTIGSIVIIGLPLVESWDTIKSVCMGMFTNDRVMKKLDDLNHRLRALTMAVPEAEKIYLLELEKTKKNDEEG

SEQ ID NO: 17 - protein, 721 aa, Oryza sativa
MASGVCPPAELGFGAEYYSVVNGVCSRAGSYFGGRPVLTQAVGYAVVLGFGAFFALFTSFLVWLEKRYVGSQHTSEWFNTAGRSVKTGLIASVIVSQWTWAATILQSSNVAWQYGVSGPFWYASGATIQVLLFGVMAIEIKRKAPNAHTVCEIVRARWGTPAHLVFLTFCLLTNVIVTAMLLLGGSAVVNALTGVNVYAASFLIPLGVVVYTLAGGLKATFLASYIHSVVVHAVLVVFVFLVYTSSSKLGSPRVVYDRLMAVASAARDCSADLSRNGQACGPVAGNFKGSYLTMLSSGGLVFGIINIVGNFGTVFVDNGYWMSAIAARPSSTHKGYLLGGLVWFAVPFSLATSLGLGALALDLPLTAAEAAKGLVPPATATALMGKSGSVLLLTMLFMAVTSAGSAELVAVSSLCTYDIYRTYLNPGASGKQILRVSRAVVLGFGCFMGVLAVVLNVAGVSLGWMYLAMGVIVGSAVIPIALLLLWSKANAVGAMGGAVSGCALGVAVWLTVAKVQYGRVNLDTTGRNAPMLAGNLVSILVGGAVHAACSLLRPQHYDWGTSREMITTVESVHAALDDELKEERLVHAKRWIVRWGLVFTAVIVVAWPALSLPARRYSLGYFTLWAAVAIAWGTVGSVVIILLPVAESWTTITKVCAGMFTNDAVYDRLDDVNLRLRAIMGAMPEAEKRYRQLHETEMHPAGTHPANDDDDDNNNNQMMHS